(19) United States
(12) Patent Application Publication
(10) Pub. No.: US 2010/0185113 A1
(43) Pub. Date: Jul. 22, 2010

(54) Coordinating System Responses Based on an Operator's Cognitive Response to a Relevant Stimulus and to the Position of the Stimulus in the Operator's Field of View

(75) Inventors: Mark A. Peot, Chapel Hill, NC (US); Mario Aguilar, Cary, NC (US)

Correspondence Address: Teledyne Technologies Incorporated, c/o Eric Gifford, 11770 E. Calle Del Valle, Tucson, AZ, 85749, US

(73) Assignee: Teledyne Scientific & Imaging, LLC

(21) Appl. No.: 12/356,681

(22) Filed: Jan. 21, 2009

(51) Int. Cl. A61B 5/0476; A61B 5/00

(52) U.S. Cl. 600/544; 600/558

(57) ABSTRACT

Neurophysiological responses such as EEG signals of brainwave activity, dilation signals of pupillary response, dwell time signals of eye movement and imaging signals of vascular response are monitored as a correlate of the operator's cognitive response. These responses are processed to determine if there is a significant cognitive response to a stimulus (visual or non-visual) to generate a positive cue. The operator's eye movement is monitored to determine when the operator fixates on the stimulus and the position of the stimulus in the operator's field-of view (FOV). The positive cue and position of the stimulus, and typically the time-stamp of the cue are output triggering a system response. The temporal sequence of cues and stimulus position may be processed to reinforce or reject the cue or refine the stimulus position.

### BACKGROUND OF THE INVENTION

1. Field of the Invention

This invention relates to cueing a system response based on an operator's cognitive response to a relevant stimulus coordinated with the position of the stimulus in the operator's field-of-view.

2. Description of the Related Art

A person's cognitive responses may be monitored to study human neurophysiology, perform clinical diagnosis and to detect significant responses to task-relevant or environmental stimuli. In the latter, the detection of such a response may be fed back or used in some manner in conjunction with the task or environment. For example, the response could be used in a classification system to detect and classify visual, auditory or information stimuli, a warning system to detect potential threats, a lie detector system etc. The detection of a significant cognitive response does not classify the stimulus but generates a cue that the operator's neurophysiology has responded in a significant way.

Various techniques for monitoring neurophysiological responses as a correlate to cognitive responses include electroencephalography (EEG), pupil dilation and blood flow or oxygenation, each of which has been correlated to changes in neurophysiology. U.S. Pat. No. 6,090,051 suggests subjecting a subject's pupillary response to wavelet analysis to identify any dilation reflex of the subject's pupil during performance of a task. A pupillary response value is assigned to the result of the wavelet analysis as a measure of the cognitive activity. Functional Near-Infrared spectroscopy (fNIRS) is an optical technique for measuring blood oxygenation in the brain. fNIRS works by shining light in the near infrared part of the spectrum (700-900 nm) through the skull and detecting how much the remerging light is attenuated. How much the light is attenuated depends on blood oxygenation and thus fNIRS can provide an indirect measure of brain activity.

In EEG systems, electrodes on the scalp measure electrical activity of the brain. The EEG signals contain data and patterns of data associated with brain activity. A classifier is used to analyze the EEG signals to infer the existence of certain brain states. In US Pub No. 2007/0185697 entitled “Using Electroencephalograph Signals for Task Classification and Activity Recognition” Tan describes a trial-averaged spatial classifier for discriminating operator performed tasks for EEG signals. Recent advances in adaptive signal processing have demonstrated significant single trial detection capability by integrating EEG data spatially across multiple channels of high density EEG sensors (L. Parra et al, “Single trial Detection in EEG and MEG: Keeping it Linear”, Neurocomputing, vol. 52-54, June 2003, pp. 177-183, 2003 and L. Parra et al, “Recipes for the Linear Analysis of EEG”, NeuroImage, 28 (2005), pp. 242-353)). The linear (LDA) classifier provides a weighted sum of all electrodes over a predefined temporal window as a new composite signal that serves as a discriminating component between responses to target versus distractor stimuli.

A rapid serial visual presentation (RSVP) system for triaging imagery is an example of a single-event EEG system (A. D. Gerson et al “Cortical-coupled Computer Vision for Rapid Image Search”, IEEE Transaction on Neural Systems and Rehabilitation Engineering, June 2006). Image clips are displayed to the analyst at a rate of approximately 10 per second and a multi-channel LDA classifier is employed to classify the brain response to the presentation of each image. If a significant response is indicated, the system flags the image clip for closer inspection. In U.S. Pub. No US 2007/0236488 entitled “Rapid Serial Visual Presentation Triage Prioritization Based on User State Assessment”, Mathan supplemented the user's EEG response with a measure of the user's physical state such as head orientation, eye blinks, eye position, eye scan patterns and posture or cognitive state such as attention levels and working memory load to further refine image triage. Head orientation and eye activity provide a way to determine whether the user is likely to perceive information presented on the screen. Images associated with a user EEG response processed during optimal user states are assigned the highest priority for post triage examination.

A helmet mounted display is a device used in some modern aircraft, especially combat aircraft. The device projects information similar to that of heads up displays (HUD) on an aircrew's visor or reticle, thereby allowing him to obtain situational awareness and/or cue weapons systems to the direction his head is pointing. The pilot can direct air-to-air and air-to-ground weapons seekers or other sensors to a target merely by point his head at the target and actuating a switch via HOTAS (hands on throttle-and-stick) controls. In close combat prior to helmet mounted displays, the pilot had to align the aircraft to shoot at a target. These devices allow the pilot to simply point his head at a target, designate it to weapon and shoot.

### SUMMARY OF THE INVENTION

The present invention provides system response based cueing on an operator's cognitive response to a stimulus coordinated with the position of the stimulus in the operator's field-of-view.

This is accomplished by monitoring an operator's neurophysiological responses such as EEG signals of brainwave activity, dilation signals of pupillary response, dwell time signals of eye movement and imaging signals of vascular response as a correlate of the operator's cognitive response to stimuli. One or more of these responses (signals) are processed to determine if there is a significant cognitive response to a stimulus (visual or non-visual) to generate a positive cue. The operator's eye movement is monitored to determine when the operator fixates on the stimulus and the position of the stimulus in the operator's field-of view (FOV). If the stimulus is visual, fixation will precede the cognitive response, hence the processing of responses (signals) can be synchronized to fixation to improve classification. The positive cue and position of the stimulus, and typically the time-stamp of the cue are output triggering a system response. The temporal sequence of cues and stimulus positions may be processed to enforce or reject the cue or refine the stimulus position or time-stamp.

In a combat environment, upon the occurrence of the positive cue the triggered response may be to slew a weapon system(s) or direct multiple operators to point at the stimulus position in a show of force. A control system at a remote location may receive cues and position data from multiple operators and synthesize the data to trigger a coordinated response to address one or more perceived stimuli. The coordinated response may be to position different weapons and operators in a show of force against different stimuli, to re-route the path of operators through a combat zone or to retask weapon systems. The positive cue and stimulus position could be provided as one input to an automated target recognition (ATR) classifier. The positive cue and stimulus position could be recorded with any triggered response as part of an archive. The archive could be used for after action reports, offline training of operators or training of automated classifiers.

These and other features and advantages of the invention will be apparent to those skilled in the art from the following detailed description of preferred embodiments, taken together with the accompanying drawings, in which:

### BRIEF DESCRIPTION OF THE DRAWINGS

FIG. 1 is a flow diagram for cueing a response based on an operator's cognitive response to a stimulus coordinated with the position of the stimulus in the operator's FOV;

FIG. 2 is a diagram of a helmet-mounted device and IR sensors for monitoring an operator's cognitive responses;

FIG. 3 is a hardware block diagram of the system;

FIG. 4 is a block diagram of a decision level classifier that fuses EEG, pupillary, dwell time and vascular neurophysiological responses to a stimulus to generate a cue;

FIG. 5 is a plot of neurophysiological responses including EEG, dwell time, fNIRS and pupil dilation signals, the EEG classifier output and an eye movement signal for fixation;

FIG. 6 is a block diagram of a spatio-temporal EEG classifier for determining the occurrence of a positive cue from a visual stimulus;

FIG. 7 is a diagram illustrating the real-time slewing of a weapon in response to the positive cue and position of the stimulus; and

FIG. 8 is a diagram illustrating the coordinated response of soldiers and weapons to cues and position data provided by multiple operators.

### DETAILED DESCRIPTION OF THE INVENTION

The present invention provides system response based cueing on an operator's cognitive response to a stimulus coordinated with the position of the stimulus in the operator's field-of-view. Response based cueing may be used in a wide variety of consumer, security and warfare environments in which relevant stimuli produce strong cognitive responses. Without loss of generality, our approach will be presented for a warfighter (operator) in an urban combat environment. In this environment, stimuli (visual or non-visual) occur randomly or asynchronously. In other applications, the presentation of stimuli may be controlled or known.

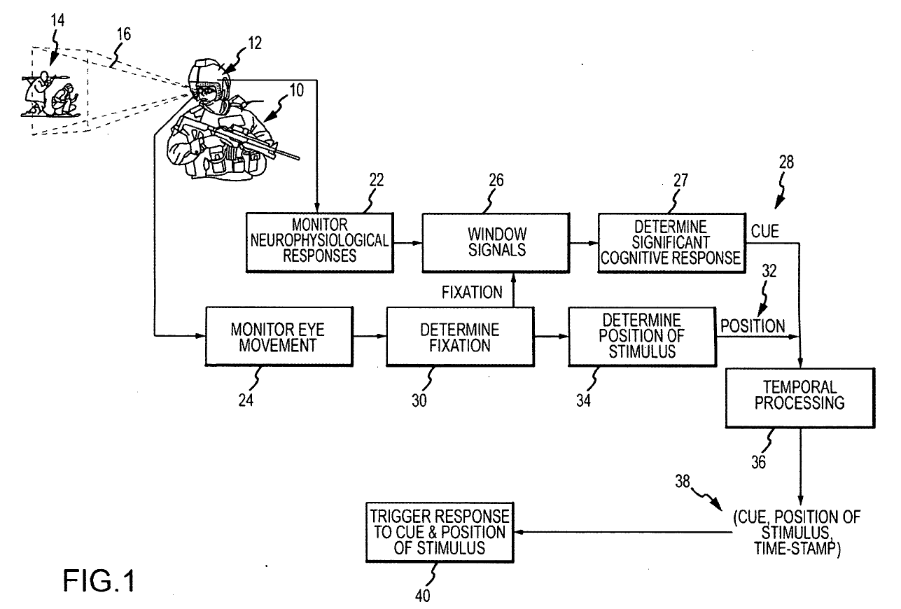

As shown in FIG. 1, a warfighter 10 outfitted with a helmet mounted device (HMD) 12 for monitoring, classifying and transmitting cues based on the warfighter's cognitive responses to stimuli is on patrol in an urban combat environment. The HMD is also configured to monitor eye movement, determine fixation and output the position of a relevant stimulus 14 in the warfighter's field-of-view (FOV) 16. Relevant stimuli can be any visual or non-visual stimuli that trigger a strong cognitive response. For example, a terrorist with a rocket propelled grenade (RPG), an explosion or an odor are examples of relevant visual and non-visual stimuli. Some stimuli may trigger a strong response but are not relevant to the task. For example, children chasing a camel down the street or the sound of a car back firing. Other stimuli may only trigger weak responses. The position of the stimulus may be output as the line-of-sight (LOS) with respect to the warfighter, the LOS and range or the geo-location (GPS coordinates) of the stimulus.

As the warfighter 10 scans the environment, HMD 12 monitors the warfighter's neurophysiological responses (step 22) and eye movement (step 24). The neurophysiological responses may include one or more of EEG signals of brainwave activity, dilation signals of pupillary response, dwell time signals of eye movement and imaging signals of vascular response as a correlate of the warfighter's cognitive response. These responses (signals) are time windowed (step 26) and processed (step 27) to determine if there is a significant cognitive response to a relevant stimulus to generate a positive cue 28. The warfighter's eye movement is monitored to determine when the warfighter fixates on the stimulus (step 30) and the position 32 of the stimulus in the warfighter's field-of view (FOV) (step 34). If the stimulus is visual, fixation will precede the cognitive response, hence the time-window of signals in step 26 can be synchronized to fixation to improve classification. If the stimulus is non-visual, the natural response of the warfighter is to turn and fixate on the perceived position from which the stimulus originates.

The temporal sequence of cues and stimulus positions may be processed (step 36) to enforce or reject the cue or refine the stimulus position or time-stamp. For example, if the stimulus represents a real threat the warfighter will tend to dwell on the stimulus and engage higher cognitive processes to respond to the threat. If a relevant stimulus is moving, the warfighter will tend to follow the stimulus in what is known as “smooth pursuit”. Conversely, if the stimulus is a false alarm the cue will diminish rapidly and the warfighter will continue to scan in a more random manner.

The positive cue and position of the stimulus, and typically the time-stamp of the cue are output (step 38) triggering a system response (step 40). For example, the HMD will transmit positive cues, stimulus position and a time stamp wirelessly to a remote location. The response may be triggered in “real time” e.g. permitting an immediate and timely response to the stimulus or threat, quasi real time e.g. with some delay but in reaction to the stimulus, or offline.

In the urban combat environment, upon the occurrence of the positive cue, the triggered system response may be to slew a weapon system(s) or direct multiple operators to point at the stimulus position in a show of force. A control system at a remote location may receive cues and position data from multiple operators and synthesize the data to trigger a coordinated response to address one or more perceived stimuli and enhance situational awareness. The coordinated response may be to position different weapons and operators in a show of force against different stimuli, to re-route the path of operators through a combat zone, to retask weapon systems, or to provide the entire group a more complete picture of a situation. The positive cue and stimulus position could be provided as one input to an automated target recognition (ATR) classifier. The positive cue and stimulus position could be recorded with any triggered response as part of an archive. The archive could be used for after action reports, offline training of operators or training of automated classifiers.

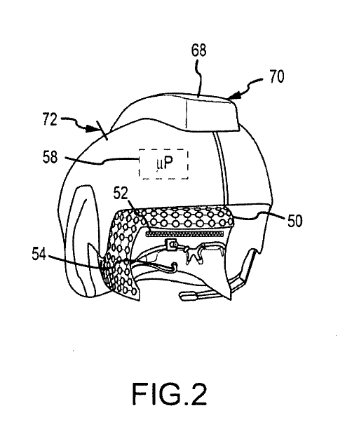

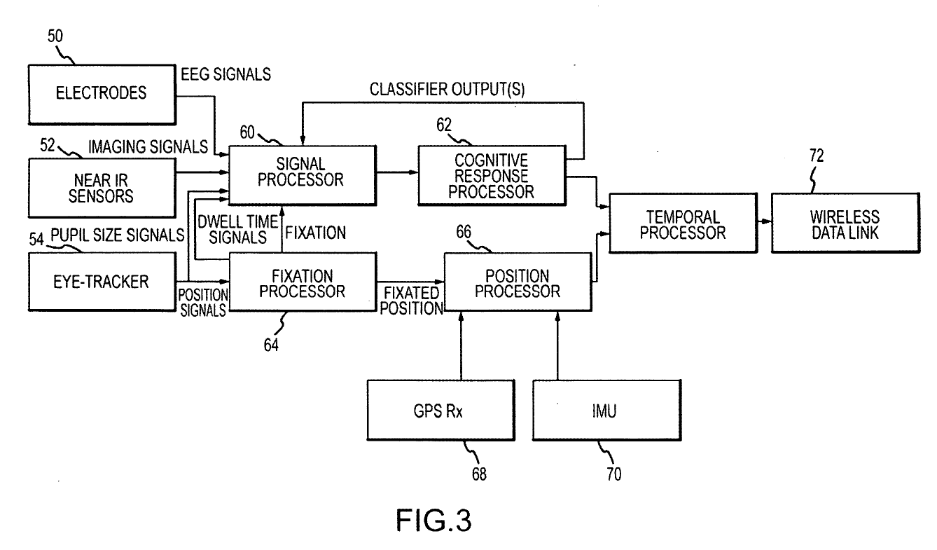

An embodiment of HMD 12 is depicted in FIGS. 2 and 3. In this configuration, the HMD includes electrodes 50 placed on the warfighter's scalp to generate multiple spatial channels of EEG signals, each spatial channel including a high-resolution temporal signal typically representative of an amplitude difference between a pair of electrodes. Near IR sensors 52 are suitably placed on the warfighter's forehead. The sensors shine light in the near IR part of the spectrum (700-900 nm) through the skull and detect the attenuation of the reemerging light. The degree of attenuation depends on blood oxygenation and thus NIRS provides imaging signals as a correlate of brain activity. An eye-tracker 54 measures the instantaneous position of the eyes by detecting the pupil (as the detection of light reflected off the back of the retina due to the NIR light projected onto the eye). The measure of the diameter provides the pupil size signals. The measure of the position of the eyes provides the position signals. With the position sampled at high rates, one can determine the instantaneous displacement. If the displacement, measured as a change in position or derivatives such as the velocity, surpasses a reasonable small threshold, it means that the eyes are moving. A resumption of the stable position indicates a fixation. The persistence of fixation provides a dwell time signal. Other configurations may include a subset of these sensors or other sensors that measure different neurophysiological responses as a correlate of cognitive response.

Although it is understood that all processing could be integrated into a single processor 58 as shown in FIG. 2 or allocated among a plurality of processors in a variety of ways, for clarity signal processing is divided among several functional processors in FIG. 3. A signal processor 60 pre-processes the raw neurophysiological response data to segment the signals into time windows, reduce noise etc. In a continuous environment in which relevant stimuli may occur at any time, the signals are typically segmented in overlapping windows to position the cognitive response within a window for classification. The time windowing may be uniform across all modalities or tailored to the time scale of each modality. As dwell time is a measure of the persistence of fixation windowing is not relevant. The pre-processing function may vary with the modality. For example, the dilation signals may be processed to remove “blink” artifacts. A cognitive response processor 62 fuses the different modalities of neurophysiological responses (signals) to determine if there is a significant cognitive response to a stimulus to generate a positive cue for a particular window. The cue may be binary valued (0/1) or continuous ([0,1]) and time-stamped. Fusion may occur at the data, feature or decision levels.

A fixation processor 64 monitors the position signals to first determine fixation on a particular stimulus and to provide the dwell time signal. Fixation occurs when the eyes remain focused on a constrained spatial region of, for example, less than half a degree. A position processor 66 receives the position signals for the left and right eyes for a fixated position and determines both the LOS and range to the stimulus position. Range can be computed based on the ‘vergence’ of the two eyes e.g. where they focus in space. This gives position with respect to the warfighter. If the absolute position e.g. geo-location in GPS coordinates, is required, a GPS receiver 68 provides the GPS coordinates of the warfighter and an inertial measurement unit (IMU) 70 provides the orientation of the HMD e.g. yaw, pitch and roll. From this information the position processor can determine the geo-location of the stimulus. The processor outputs the position and time-stamp.

At this point, the system marries the cue and position information to output the cue, position and time stamp. The system will typically generate this triplet for each window in an ongoing time sequence. The entire data stream or only the positive cues may be output via wireless data link 72.

Alternately, the system may be configured to process the temporal cues and fixation measurements to reinforce or reject the positive cue and refine the stimulus position. The use of dwell time as a correlate of cognitive response is one approach. Similarly, the cue generated by the individual or fused classifiers in the cognitive response processor may be fed back as features to one or more of the classifiers. These approaches treat the temporal properties of fixation and classifier response independently. A temporal processor 74 could be configured to look at the temporal sequence of the paired cue and fixation. A relevant stimulus should produce individual and fused classifier outputs that build quickly to a maximum and than falls off as the warfighter's brain engages high level processes to engage the threat with persistent fixation on the stimulus. Although the strong cognitive response is typically momentary the overall temporal response is distinguishable from a non-relevant stimulus. First, the high level processes required to engage the threat will present differently than a return to scanning with no threat. Second, the warfighter will remain fixated on the threat for some time. If the stimulus is moving, temporal processing can identify the “smooth pursuit” of eye movement to reinforce the positive cue and track the position of the stimulus. Temporal processing should reinforce the accurate detection of positive cues and improve the rejection of false alarms. Furthermore, depending on the time resolution of the overlapping windows, temporal processing can refine both the time-stamp and position of the relevant stimulus.

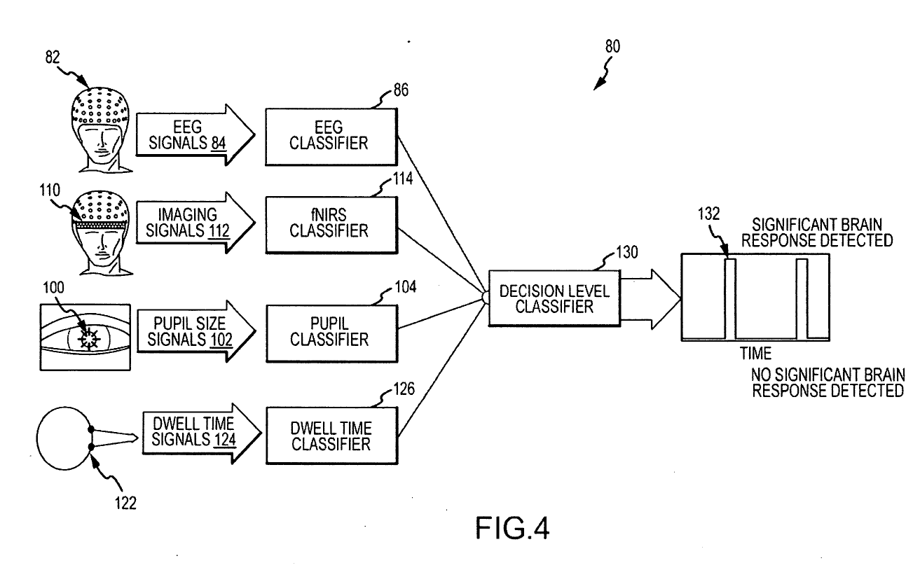

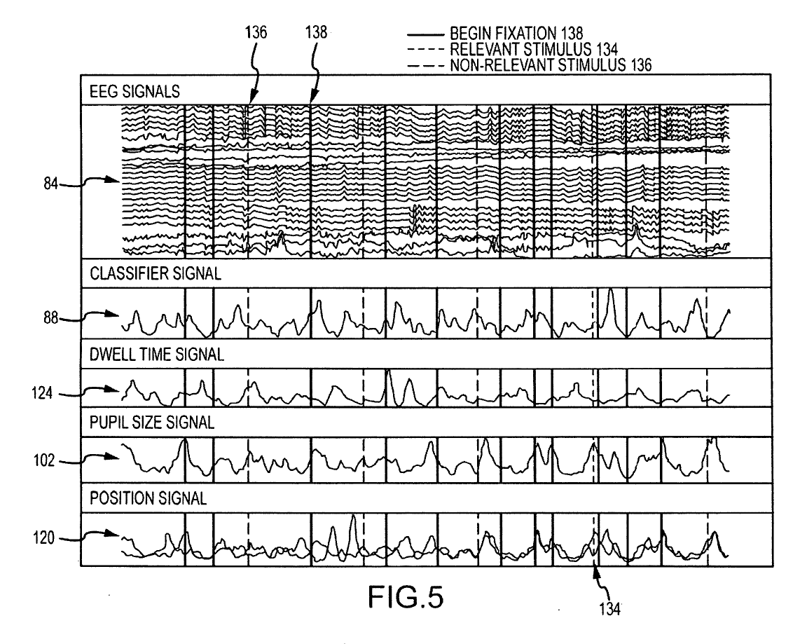

An embodiment of a fused-modality classifier 80 and the relevant signals is shown in FIGS. 4 and 5.

This particular system is configured to detect significant cognitive responses to “single-event” stimuli e.g. a stimulus that may occur only once. The single-event stimulus and the warfighter's cognitive response to that stimulus may persist for some time but the initial event that caused the warfighter to respond is singular. For example, if a warfighter sees a terrorist with an RPG, the terrorist and RPG persist but the event of first seeing the terrorist that triggers the response is singular. In other words, the event is the warfighter's seeing the terrorist not the existence of the terrorist. Similarly, if a warfighter responds to a sudden noise and turns to see a threat there may be two separate single-event stimuli, the noise and then the visual threat. The approach may also be used in applications in which the relevant stimulus is repeated allowing for trial-averaging to suppress unrelated stimuli and improve SNR.

This particular system also employs decision level fusion of the different modalities. Feature level fusion is also possible. Depending upon the application, the individual modality classifiers and/or decision level classifier can be configured and trained to either detect all significant cognitive responses or to only detect significant cognitive responses caused by particular stimuli. In the former case all stimuli that produce a significant cognitive response are “relevant stimuli” whereas in the latter case only the particular stimuli are relevant. In the former case, the application may not care what stimuli caused the strong response. Alternately, the classifier may be constructed to detect all significant cognitive responses and use the temporal post-processing to eliminate or reduce the false alarms. In the latter case, maybe only some of the individual classifiers are constructed and trained to differentiate relevant from non-relevant stimuli while the other detect all strong stimuli. For example, maybe EEG and pupil dilation signals are rich enough to perform such differentiation while the NIRS and dwell time signals only support the gross classification.

### EEG Classifier

Electroencephalography (EEG) is the neurophysiologic measurement of the electrical activity of the brain recording from electrodes 82 placed on the scalp of the warfighter. The EEG signals 84 contain data and patterns of data associated with brain activity. A multi-channel spatial classifier 86 analyzes the EEG signals to detect significant brain responses to task-relevant stimuli. The integration of EEG data spatially across multiple channels improves the SNR much like trial-averaging.

The classifier can, for example, be constructed to extract features (e.g. time domain such as amplitude and/or frequency domain such as power) from one or more time windows and render a likelihood output 88 (continuous value from 0 to 1) or decision output (binary value of 0 or 1) based on a weighted (linear or non-linear) combination of the features. Typical classifiers include the LDA, support vector machine (SVM), neural networks or AdaBoost. A rich set of features may be available from which a smaller subset of features are selected for a particular application based on training. The classifier is trained based on the extracted features to detect a significant brain response for a single-event relevant stimulus. The classifier may be trained to recognize any significant brain response or, more typically, it may be trained to recognize significant brain response for particular relevant stimuli and reject significant grain responses for non-relevant stimuli.

### Pupil Classifier

Pupil response provides a direct window that reveals sympathetic and parasympathetic pathways of the autonomic division of the peripheral nervous system. Task-evoked pupil dilations are known to be a function of the cognitive workload and attention required to perform the task. It has long been known that the pupil dilates in response to emotion evoking stimuli. Thus, cognitive task related pupillary response provides a modality that can be used to detect significant brain responses to single-trial task-relevant stimulus. Because the EEG and pupil responses are associated with different parts of the nervous system, specifically the brain area that triggers the pupillary response is deep inside the brain and thus not measurable by EEG electrons on the scalp, we hypothesized that the two could be complementary and that fusing the EEG and pupil classifiers would improve classification confidence. See co-pending U.S. application Ser. No. 11/965,325 entitled “Coupling Human Neural Response with Computer Pattern Analysis for Single-Event Detection of Significant Brain Responses for Task-Relevant Stimuli” filed Dec. 27, 2007, which is hereby incorporated by reference.

A camera such as an EyeLink 1000 video based eye tracking device is trained on the operator's pupil 100 to monitor pupil activity e.g. size, continuously over time. The recording of pupil size signals 102 is synchronized with EEG data acquisition. When presented with baseline stimulus or a distractor, pupil activity is fairly flat. However, when presented with a task-relevant stimulus, pupil activity indicates a fairly dramatic change. The pupil data is passed to pupil classifier 104 where it is pre-processed (e.g. remove blinks), spatio-temporal pupil features extracted and fused and then classified to generate a binary decision pupil output or continuous likelihood pupil output (not shown). The classification algorithm can be selected from LDA, ARTMAP, and RVM etc. A subset of features can be selected during training for a particular application. Alternately, Marshall's wavelet approach may be used to detect significant brain response to relevant stimuli.

fNIRS Classifier

Functional near-infrared spectroscopy (fNIRS) is a type of functional neuroimaging technology that offers a relatively non-invasive, safe, portable, and low-cost method of indirect and direct monitoring of brain activity. By measuring changes in near-infrared light, it allows researchers to monitor blood flow in the front part of the brain. More technically, it allows functional imaging of brain activity (or activation) through monitoring of blood oxygenation and blood volume in the pre-frontal cortex. It does this by measuring changes in the concentration of oxy- and deoxy-haemoglobin (Hb) as well as the changes in the redox state of cytochrome-c-oxidase (Cyt-Ox) by their different specific spectra in the near-infrared range between 700-1000 nm.

The functional near-infrared spectroscopy (fNIRS) sensor 110 is attached to the operator's forehead and generates imaging signals 112 as a measure of light attenuation as the operator is exposed to stimuli in the environment. A fNIRS classifier 114 extracts features to analyze the signals for changes in the blood flow or oxygenation levels of the brain before, during, and after the stimulus and generates either a decision or likelihood output (not shown).

### Dwell Time Classifier

How long an operator remains fixated on a stimulus is another possible correlate to brain activity. If the operator's “dwell time” on a stimulus is long that is an indicator of significant cognitive response to a relevant stimulus. Conversely, if the operator does not fixate on a stimulus or only fixates momentarily and resumes scanning that is a counter indicator of a significant cognitive response.

An eye-tracker measures a pupil position signal 120 for each eye 122. The operator has ‘fixated’ on a stimulus when the position signals are constrained to a small region of visual space (e.g. motion is less than a fraction of a degree). As the operator remains fixated on or re-enters a previously fixated stimulus, the dwell time 124 increases until the operator looks in another direction. A dwell time classifier 126 maps the dwell time 124 to either a binary decision output or a continuous likelihood output (not shown).

### Fusion Level Classifier

The fusion of complementary modalities enhances the detection of significant brain responses to relevant performance. Each modality generates an output indicative of the brain response and the decisions or likelihoods for the different modalities are then fused. Decision-level fusion is particularly effective for combining these modalities. Each modality classifier's likelihood output is mapped to a binary decision output and these 0/1 “decisions” are fused. A feature-level fusion classifier such as another LDA that accepts the likelihood outputs of the individual classifiers may also be implemented.

The decision-level classifier 130 is implemented to achieve an optimal combination of maximum likelihood estimates achievable between the complementary decisions. An effective approach is to use Bayesian inference where the modality classifiers' binary decisions are treated as multiple hypotheses that need to be combined optimally. For this approach to be effective the different modalities must be complementary, not merely redundant. The decision level classifier optimally fuses the four decisions based on the EEG, pupil, fNIRS and dwell time modalities according to the operating points on their receiver operating characteristic (ROC) curves at which each of the decisions were made with certain probability of detection and probability of false alarm to generate a final binary decision 132 as to the cognitive response state. Training data is used to obtain the ROC curves and choose the operating points associated with the EEG, fNIRS, pupillary, dwell time and decision-level classifiers.

As shown in FIG. 5, signals at or near the relevant stimulus 134 represent neurophysiological activity during performance of natural tasks (e.g., observing and/or manipulating the environment). Signals associated with a response following the occurrence of stimulus 134 will present a deviation from this natural neurophysiological activity and will be detected as an anomaly by the proposed signal classifier. The characteristics of the signal in each of the different modalities (i.e., EEG, pupil, fNIRS) are learned by the signal classifier module and are differentiated from those present during normal activity. The occurrence of non-relevant stimuli 136 and the operator's fixation 138 to different stimuli are also shown.

### Spatio-Temporal EEG Classifier

The brain response to stimuli is not a stationary pulse. The brain response reflects neurophysiological activities located in selectively distributed sites of the brain evolving with a continuous time course. In human operators, the first indication of brain response to a stimuli occur approximately 80 ms after the onset of the stimuli and may continue for up to approximately 900 ms-1.5 sec as the signal propagates through different areas of the brain.

The brain response to “relevant” information is a non-stationary signal distributed across multiple areas of the brain. Specifically, perceptual information from the senses is first processed in primary sensory cortex from where it travels to multiple cortical mid-section areas associated with separately processing the spatial (“Where”) and semantic (“What”) meaning of the information. The resulting information patterns are matched against expectations, relevance or mismatch at which point signals are relayed to more frontal regions were higher-level decisions can be made about the relevance of the information. If enough evidence exists, a commitment to respond is then made. This suggests that the decision process involves multiple sites (space) across a relative long time window (and time). Conventional EEG classifiers only process data captured at a certain critical time period after stimulus onset. Our approach to analyzing the EEG signal as detailed in co-pending application Ser. No. 11/965,325 attempts to capture this spatio-temporal pattern by collecting evidence of this non-stationary signal and combining it to improve detection confidence.

In this particular example, we assume that the relevant stimulus is visual, hence fixation exists prior to the occurrence of any significant cognitive response. Consequently, processing (e.g. windowing) of the EEG signals can be synchronized to the onset of fixation.

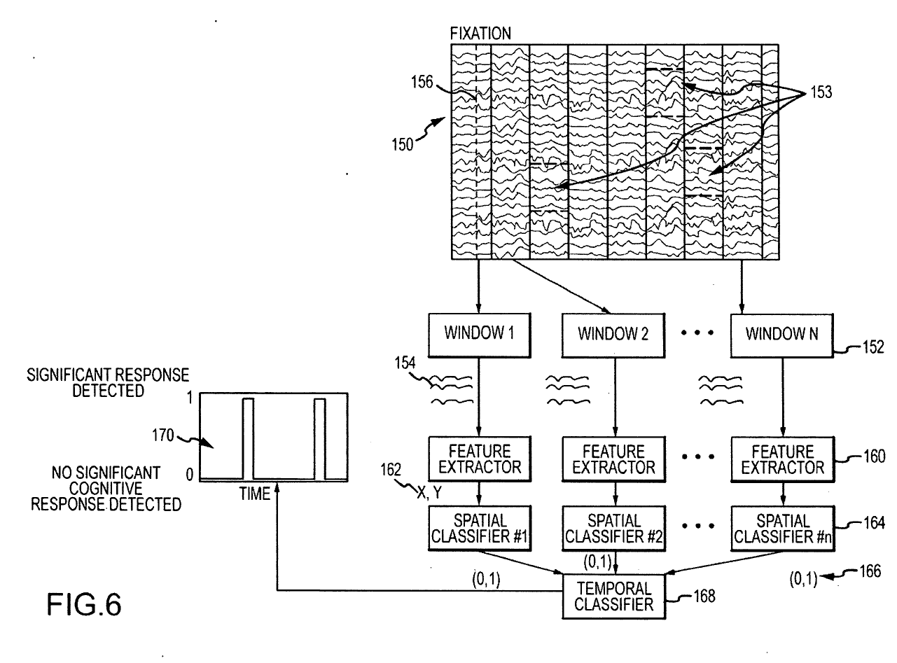

As shown in FIG. 6, the multiple channels of EEG data 150 are subdivided into a plurality of windows 152 sufficient to capture the temporal evolution of the brain response to a stimulus. Each spatial channel includes a temporal signal 153 typically representative of an amplitude difference between a pair of electrodes. Each window contains a different temporal segment of data 154 from the onset of an operator's fixation 156 on a relevant stimulus for a subset, typically all, of the spatial channels.

In order to detect temporal patterns across the different time windows it is useful to control four separate parameters; the window duration, the number of windows, the total temporal window captured and the overlap between windows. The window duration and overlap are typically uniform but could be tailored based on specific training for certain applications. Window duration may be in the range of 20-200 ms and more typically 50-100 ms; long enough to capture signal content with sufficient SNR yet short enough to represent a distinct portion of the non-stationary signal. The number of windows must be sufficient to provide a robust temporal pattern. The total temporal window typically spans the onset of the fixation on the stimuli to a threshold window beyond which the additional data does not improve results. The threshold may be assigned based on the response of each operator or based on group statistics. The threshold window for most operators for our experimental stimuli is near 500 ms. Window overlap is typically 25-50%, sufficient to center critical brain response transitions within windows and to provide some degree of temporal correlation between spatial classifiers. Larger overlaps may induce too much correlation and become computationally burdensome.

Feature extractors 160 extract features X, Y, . . . 162 from the respective windows of EEG data. These features may be time-domain features such as amplitude of frequency-domain features such as power or combinations thereof. The extracted features may or may not be the same for each window. To optimize performance and/or reduce the computational load, the nature and number of features will be determined during classifier training, typically for a particular task-relevant application. For example, classifier training may reveal that certain features are better discriminators in early versus late windows. Furthermore, since the temporal evolution of the signal roughly corresponds to its propagation through different areas of the brain features may be extracted from different subsets of spatial channels for the different windows. Training would identify the most important spatial channels for each window.

Once extracted, the features from the different temporal windows are presented to respective spatial classifiers 164. Each classifier is trained based on the extracted features for its particular window to detect a significant brain response for a task-relevant stimulus. The classifier may be trained to recognize any significant brain response or, more typically, it may be trained for a particular task and stimuli relevant to that task. Brain activity is measured and recorded during periods of task relevant and irrelevant stimulation and the classifiers are trained to discriminate between the two states. Specific techniques for training different classifiers are well known in the art. A linear discrimination analysis (LDA) classifier of the type used in single-window RSVP systems was configured and trained for each of the N spatial classifiers. The LDA classifier described by Parra linearly combines the multiple spatial EEG channels to form an aggregate representation of the data. Other linear and non-linear classifiers such as support vector machines (SVM), neural networks or AdaBoost could also be employed. Different classifiers may be used for the different windows. Each classifier 164 generates a first level output 166. The classifiers may be configured to generate either a likelihood output e.g. a continuous value from 0 to 1, or a decision output e.g. a binary value of 0 or 1 depending on the type of fusion used to combine the outputs.

The spatial classifiers' first level outputs are presented to a temporal fusion classifier 168 that combines them to detect temporal patterns across the different time windows relating to the evolution of the non-stationary brain response to task-relevant stimulus and to generate a second level output 170 indicative of the occurrence or absence of the significant non-stationary brain response. In this configuration, the second level output is a binary decision as to the brain state for a current stimulus. Although there is some latency due to data collection e.g. 500 ms from the onset of the stimulus, the processing time is small, approximately 5 ms, so that the system can generate decision level outputs in real-time that keep up with the presentation or occurrence of stimuli. The decision level output 170 could be fused with the decision level outputs for the other modalities as described in FIG. 4.

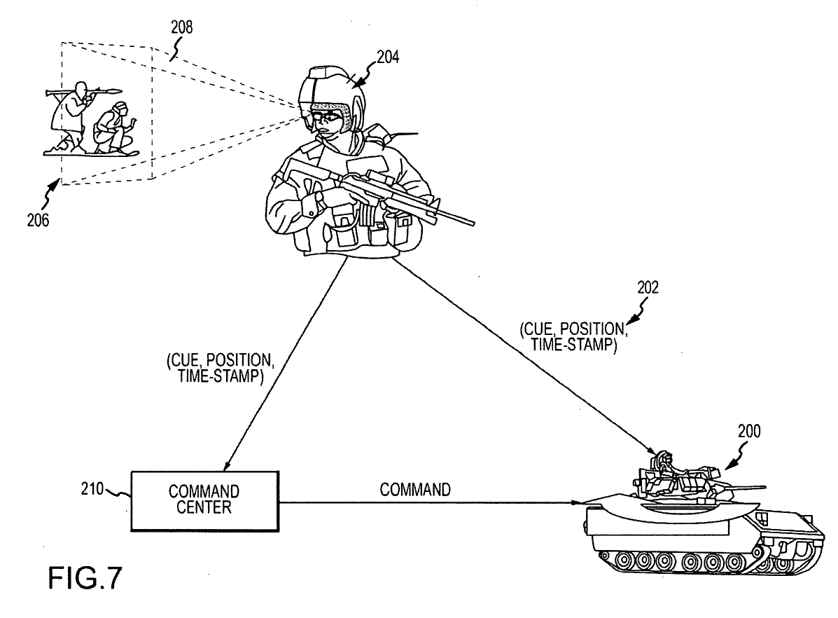

As shown in FIG. 7, in the urban combat environment, a weapons system 200 is slaved to the cognitive response based cue and stimulus position 202 generator by a warfighter's HMD 204 in response to a relevant stimulus 206. The warfighter fixates on the stimulus 206 in his FOV 208 allowing the HMD to compute and transmit the stimulus position along with the positive cue. In this example, the cue and position are accompanied by a time-stamp. The cue may be transmitted directly to the weapons system 200 causing it to point at the position of the stimulus in a “show of force”. Alternately, the cue may be transmitted to a command center 210 that processes the information, alone or in context with cues from other operators or other information, and issues a command to the weapons system to engage the stimulus. For example, the positive cue and stimulus position could be provided as one input to an automated target recognition (ATR) classifier at the command center or the weapons system. Control of the weapons system to engage the threat can be done in real-time without requiring the warfighter to take any affirmative action. In these types of combat situations, an immediate show of force can be very effective to dissuade the enemy.

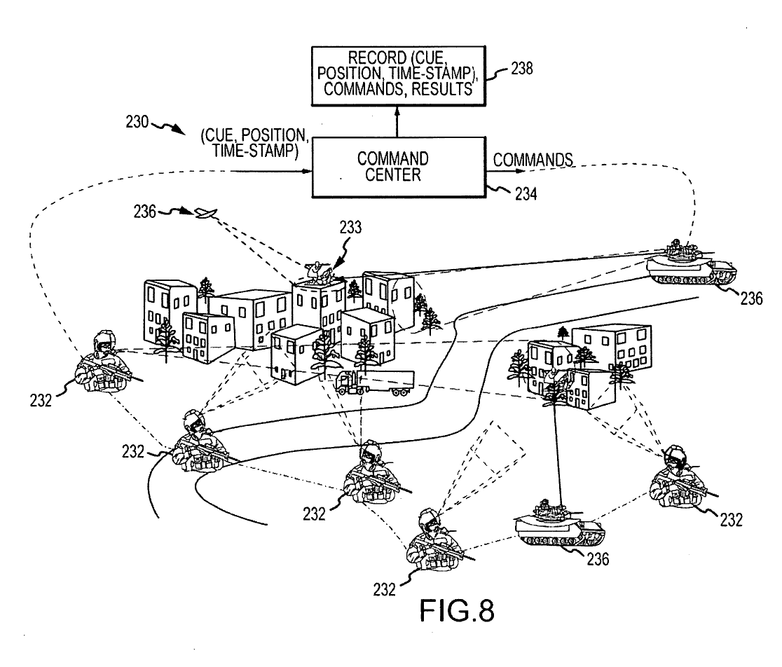

As shown in FIG. 8, in another urban combat environment, the cognitive response based cues 230 from multiple warfighters 232 to relevant stimuli 233 (e.g. terrorists with weapons) are received at a remote command center 234 and used to coordinate the response of the warfighters and different weapons systems 236. Upon the occurrence of the positive cue from one or more warfighters, the triggered response may be to slew a weapon system(s) or direct multiple warfighters to point at the stimulus position in a show of force. The command center receives cues and position data from multiple warfighters and synthesizes the data to trigger a coordinated response to address one or more perceived stimuli. The coordinated response may be to position different weapons and warfighters in a show of force against different stimuli, to re-route the path of warfighters through a combat zone or to retask weapon systems. The positive cue and stimulus position could be recorded with any triggered response as part of an archive 238. The archive could be used for after action reports, offline training of warfighters or training of automated classifiers.

Although response cueing to an operator's cognitive response coordinated with the position of the stimulus in the operator's EOV has particular applicability to military environments, it may be useful in other commercial and security applications as well. For example, a security guard could monitor a large array of video feeds. A classifier would look for a significant cognitive response and marry that positive cue to the particular feed that caused the response. The cue could be used to alert the security guard, recording systems, substations or others. In another example, response cueing could be used in conjunction with a person's watching an interactive television program or the web to cue on the presentation of certain information or products. The position and timing of the positive cue can be correlated to the programming or web content to identify the information or product and take some action. For example, more detailed information related to the stimulus could be retrieved or product or ordering information could be retrieved. In yet another example, response cueing could be incorporated into user response systems in which control groups of operators watch movies or advertisements before they are released to assess user reaction and feedback. This approach could supplement or replace other methods of user feedback and would identify the particular stimulus that is evoking a strong response. This information could be aggregated and used to reedit the advertisement or movie.

While several illustrative embodiments of the invention have been shown and described, numerous variations and alternate embodiments will occur to those skilled in the art. Such variations and alternate embodiments are contemplated, and can be made without departing from the spirit and scope of the invention as defined in the appended claims.

We claim:

1. A method of cueing a system response, comprising:
monitoring neurophysiological responses of an operator subjected to stimuli;
processing the neurophysiological responses to determine if the operator had a significant cognitive response to a stimulus to generate a positive cue;
monitoring the operator's eye movement to determine when the operator fixates on the stimulus;
determining the position of the stimulus in the operator's field-of view (FOV);
outputting the positive cue and the position of the stimulus; and
triggering a system response to the positive cue and the position of the stimulus.

2. The method of claim 1, wherein determining the stimulus position comprises:
determining the orientation of the stimulus with respect to the operator; and
determining the range of the stimulus with respect to the operator.

3. The method of claim 2, further comprising:
determining the geo-location of the operator; and
determining a geo-location of the stimulus from the geo-location of the operator and the position of the stimulus.

4. The method of claim 1, wherein the stimulus comprises a non-visual stimulus.

5. The method of claim 1, wherein the stimulus comprises a visual stimulus.

6. The method of claim 5, wherein the operator fixates on the visual stimulus prior to the operator's cognitive response to the stimulus, further comprising:
synchronizing the neurophysiological responses to the operator's fixation on the stimulus for processing; and
processing the synchronized neurophysiological responses.

7. The method of claim 6, wherein at least one window is positioned relative to the onset of fixation and the neurophysiological responses within the window processed.

8. The method of claim 7, wherein one said window is positioned to capture neurophysiological responses both before and after the onset of fixation.

9. The method of claim 7, wherein the neurophysiological responses within said at least one window are processed by:
extracting features from the neurophysiological responses, said features based on the assumption that the at least one window has a specified position relative to the onset of fixation; and
presenting the extracted features to a computer-implemented classifier trained to detect patterns of the extracted features and to generate the positive cue indicative of the occurrence a significant brain response.

10. The method of claim 1, wherein the monitored neurophysiological responses include at least one of EEG signals of brainwave activity, dilation signals of pupillary response, dwell time signals of eye movement and imaging signals of vascular response.

11. The method of claim 10, wherein at least two of the EEG, dilation, dwell time and imaging signals are monitored and processed.

12. The method of claim 1, wherein the response is triggered in real-time with respect to the operator's cognitive response to the stimulus.

13. The method of claim 1, further comprising:
wireless transmitting the output of the positive cue and position of the stimulus from the operator to a remote location where the system response is triggered.

14. The method of claim 13, wherein the system response is to aim a weapon at the position of the stimulus.

15. The method of claim 13, where the system response is to command multiple operators to turn toward the position of the stimulus in a show of force.

16. The method of claim 13, wherein a control system at the remote locations receives cues and position data from multiple operators and synthesizes the data to trigger the system response.

17. The method of claim 1, wherein the positive cue and position of the stimulus are provided as one of a plurality of inputs to classifier, the output of the classifier triggering the system response.

18. The method of claim 1, wherein the triggered system response is to record the positive cue and position of the stimulus and any action taken in response to that stimulus.

19. The method of claim 1, wherein a time sequence of cues and fixation measurements are output, further comprising processing the temporal cues and fixation measurements to reinforce or reject the positive cue and refine the stimulus position.

20. A system comprising:
a head-mounted system for an operator, said system comprising:
at least one sensor monitoring neurophysiological responses of an operator subjected to stimuli;
a classifier processing the neurophysiological responses to determine if the operator had a significant cognitive response to a stimulus to generate a positive cue;
an eye-tracking device monitoring the operator's eye movement to determine when the operator fixates on a stimulus;
means for determining the position of the stimulus in the operator's field-of view (FOV); and
a wireless data link transmitting the positive cue and the position of the stimulus; and
a remote system receiving the cue and position and triggering a system response to the positive cue and the position of the stimulus.

21. The system of claim 20, wherein said at least one sensor monitors neurophysiological responses including at least one of EEG signals of brainwave activity, dilation signals of pupillary response, dwell time signals of eye movement and imaging signals of vascular response.

22. The system of claim 20, wherein the remote system triggers a system response of aiming a weapon at the position of the stimulus or commanding multiple operators to turn toward the position of the stimulus.

23. The system of claim 22, wherein the remote system receives cues and position data from multiple operators and synthesizes the data to trigger the system response.

24. The system of claim 20, wherein the operator fixates on the visual stimulus prior to the operator's cognitive response to the stimulus, further comprising a data pre-processing system that windows the monitored neurophysiological responses into at least one time window synchronized to the operator's fixation on the stimulus.
